(19) United States
(12) Patent Application Publication
(10) Pub. No.: US 2024/0264179 A1
(43) Pub. Date: Aug. 8, 2024

(54) MARKER SET FOR EVALUATING THE DEVELOPMENT OF INFANT’S COMMUNICATION ABILITY AND USE THEREOF

(71) Applicant: Beijing Sanyuan Foods Co., Ltd., Beijing (CN)

(72) Inventors: Lijun CHEN, Beijing (CN); Minghui ZHANG, Beijing (CN); Weicang QIAO, Beijing (CN); Junying ZHAO, Beijing (CN); Ge JIA, Beijing (CN); Yan LIU, Beijing (CN); Yaling WANG, Beijing (CN); Qian LIU, Beijing (CN); Baoyu YANG, Beijing (CN)

(21) Appl. No.: 18/476,528

(22) Filed: Sep. 28, 2023

Related U.S. Application Data
(63) Continuation of application No. PCT/CN2023/101756, filed on Jun. 21, 2023.

(30) Foreign Application Priority Data
Feb. 7, 2023 (CN) 202310143580.9

(51) Int. Cl. G01N 33/92; G01N 33/68

(52) U.S. Cl. CPC G01N 33/92; G01N 33/6851; G01N 2800/38

(57) ABSTRACT

The present invention provides a marker set for evaluating the development of an infant's communication ability. The marker set is composed of the following fatty acids: C18:3n6, C18:2n6t, C20:3n3, C18:3n3, C18:2n6c, C20:4n6, C20:3n6, C22:6n3, and C20:5n3. The present invention further provides a method for evaluating the development of an infant's communication ability using the marker set. The present invention achieves the evaluation on the development of an infant's communication ability by extracting and detecting the contents of particular fatty acids in a breast milk or dairy product sample fed to an infant, and the development level of the infant among the peers can be inferred based on the evaluation result, so as to give suggestions relevant to diet and nutrition for the mother timely. The method is low in cost, simple and effective, and suitable for large-scale evaluation.

### CROSS REFERENCE

The present application claims priority to Chinese Patent Application No. 202310143580.9 filed with the China National Intellectual Property Administration on Feb. 7, 2023 and entitled “MARKER SET FOR EVALUATING THE DEVELOPMENT OF INFANT'S COMMUNICATION ABILITY AND USE THEREOF”, the contents of which are incorporated by reference herein in its entirety.

### TECHNICAL FIELD

The present invention relates to the field of detection and nutritional evaluation, and in particular, to a marker set for evaluating the development of an infant's communication ability and a use thereof.

### BACKGROUND ART

Fat, as an indispensable nutrient substance for sustaining life and a basic metabolite, is a main energy storage form in organisms. Researches have shown that the nutritive value of fat is determined by fatty acids. Breast milk is the main food for infants. The types, contents, and proportions of various nutrient elements contained in breast milk meet “gold standards” for babies. There are many types of fatty acids in breast milk, including medium-chain and medium- and long-chain saturated fatty acids: long-chain saturated fatty acids (e.g., palmitic acid), monounsaturated fatty acids (e.g., oleic acid); and polyunsaturated fatty acids (such as linoleic acid, ARA, DHA, and linolenic acid).

1000 Days in an infant's early life refers to a period from the pregnancy of the mother to 2 years of age of the infant, during which the nutritional status of the infant will influence the life-long health. The prevention of diseases during a person's adulthood should begin from the nutrition during the early life of the person. This has become an international research hotspot in the fields of food and medicine at present. An increasing number of evidences have shown that recommended values of nutrient reference intakes based on big data contribute to scientifically formulate nutrition policies and feeding standards for infants. Breast milk is the best food for infants. The world health organization recommends exclusive breastfeeding to infants under 6 months. The nutritious components of the breast milk and dynamic changes in contents thereof are the basis for speculating the nutrient requirements of infants at this stage. However, the breast milk components differ greatly between lactating women of different races and different dietary patterns. Birth cohort studies mainly focus on relationships of exposure factors such as nutritional condition, life style, and dietary habit with adverse pregnancy outcomes and birth defects, and optimization methods for effects of exposure in early life on the life-long health. In view of this, the present invention is specifically proposed.

### SUMMARY OF THE INVENTION

An objective of the present invention is to provide a marker set. By detecting the contents of each marker in the marker set in a sample, the development of the communication ability of an infant fed with the sample can be inferred so as to give timely nutritional suggestions and take nutritional interventions.

To achieve the above objective, the present invention provides a marker set for evaluating the development of an infant's communication ability. The marker set is composed of the following fatty acids: C18:3n6, C18:2n6t, C20:3n3, C18:3n3, C18:2n6c, C20:4n6, C20:3n6, C22:6n3, and C20:5n3.

In another aspect, the present invention further provides a method for evaluating the development of an infant's communication ability using the marker set. The method includes the following sequential steps:
(1) collecting a sample, and pretreating the sample to obtain a sample to be measured;
(2) measuring the contents of each marker of the marker set in the sample to be measured using gas chromatography-mass spectrometry (GC-MS);
(3) calculating a score Ya of the sample from the measured contents of each marker; and
(4) evaluating the development of an infant's communication ability based on the score Ya.

Preferably or alternatively, in step (1), the sample is pretreated by performing methyl esterification on the sample using a hydrochloric acid-methanol method.

Preferably or alternatively, the hydrochloric acid-methanol method specifically includes: adding 5 mL of a 0.5 mol/L hydrochloric acid-methyl alcohol solution into 200 μL of the sample, adding 2 mL of n-hexane and 2 mL of methyl alcohol thereinto, shaking the mixture for uniform mixing, shaking the same in a thermostatic water bath at 80° ° C. for 2 h, then cooling the obtained mixture to room temperature with running water, adding 2 mL of deionized water thereinto, shaking the mixture for uniform mixing, followed by refrigerated centrifugation at 5000 rpm for 5 min, and putting 1 mL of a supernatant into a sample bottle, thereby obtaining the sample to be measured. Preferably or alternatively, in step (2), the parameters of GC of the GC-MS are as follows: HP-88 GC column having specifications of 100 m×0.25 mm and 0.20 μm is used: a temperature rising procedure includes setting an initial temperature at 60° C., which is maintained for 5 min, increased to 160° C. at 8° C./min, then increased to 200° C. at 4° C./min and maintained for 5 min, and finally increased to 240° C. at 3 ºC/min and maintained for 5 min; a sample injector is Thermo Scientific TriPlus RSH automatic sample injector: a sample inlet temperature is 200° C.: a constant flow velocity mode is 1 mL/min: a sample injection volume is 1 μL; a split ratio is 10:1; and N₂ is used as a carrier gas.

Preferably or alternatively, in step (2), the parameters of MS of the GC-MS are as follows: an electron impact ion source mode is employed: an ion source temperature is 280° C.; a transmission line temperature is 240° C.: a quadropole temperature is 150° C.; and a quadropole mass scan range is m/z 35-400.

Preferably or alternatively, in step (3), Ya is calculated by: Ya=0.115×C_{C18:3m6}−1.001×C_{C18:2n6t}+0.054×C_{C20:3n3}+0.004×C_{C18:3n3}+0.003×C_{C18:2n6c}+0.065×C_{C20:4n6}+0.03×C_{C20:3n6}+0.009×C_{C22:6n3}+0.003×C_{C20:5n3}, where the concentrations of each marker are in units of ppm.

### Beneficial Effects

The present invention achieves the evaluation on the development of an infant's communication ability by extracting and detecting the contents of particular fatty acids in a breast milk or dairy product sample fed to an infant, and the development level of the infant among the peers can be inferred based on the evaluation result, so as to give suggestions relevant to diet and nutrition for the mother timely. The method is low in cost, simple and effective, and suitable for large-scale evaluation.

### BRIEF DESCRIPTION OF THE DRAWINGS

FIG. 1 is a total ion chromatogram of a methyl ester standard sample of 37 fatty acids; and

FIG. 2 is a total ion chromatogram of 37 fatty acid methyl esters of No. 10256 breast milk sample.

### DETAILED DESCRIPTION

To facilitate the understanding of the present invention, the present invention will be described more fully and in detail below with reference to the accompanying drawings and preferred experimental examples, but the protection scope of the present invention is not limited to the following specific examples.

Unless otherwise defined, all technical terms used hereinafter have the same meanings as commonly understood by those skilled in the art. The technical terms used herein are for the purpose of describing specific examples, and are not intended to limit the protection scope of the present invention.

Unless otherwise specified, various raw materials, reagents, instruments, equipments, and the like used in the present invention are commercially available or can be prepared by existing methods.

### Example 1

The example of the present invention provides a method for evaluating the development of an infant's communication ability:

Samples for detection in the present example were breast milk. The breast milk was provided by mothers who did not smoke, had no clinical signs of known diseases, had no record of taking antibiotics within four weeks before collection, delivered at term, and had no symptom and sign of mastitis.

When collecting, a breast pump washed in advance was used to collect 30 mL of milk from one or both breasts.

The breast milk was collected for 7 times at the following time points: starting from the day of delivery, colostrum on day 5±2 days after delivery, transitional milk on day 10±3 days, mature milk on month 2±3 days, mature milk on month 4±3 days, mature milk on month 6±3 days, mature milk on month 8±3 days, and late lactation milk on month 10±3 days. Mothers with an insufficient number of times of collection during breast milk collection were eliminated.

The breast milk was generally collected at 9 am to 11 am. All the mothers providing the breast milk were prohibited from intake of any food and liquid after 9:00, and the breast milk was collected 2 hours after the last breastfeeding. The breast milk samples should not be placed at 10° C.-30° ° C. for more than 6 hours, and were shaken well before detection.

The collected breast milk samples were placed in a disinfection container and preserved at −20° C. Apart from collecting the breast milk samples, informations, such as the age, height, 24-hour dietary recall, dietary habit, and the use of vitamin and/or mineral supplements of each mother providing the breast milk, and an infant birth status, were collected.

Meanwhile, the developments of the communication ability of the infants belonging to each mother during the collection cycle of the mother were tracked in a form of after-scenario questionnaire (ASQ), and actual communication ability scores Ya of the infants corresponding to each sample were obtained.

A total of 254 samples were collected by the above method.

Before detection, each breast milk sample was pretreated by performing methyl esterification thereon using a hydrochloric acid-methanol method for subsequent further measurements. A specific implementation process was as follows:
5 mL of a 0.5 mol/L hydrochloric acid-methyl alcohol solution was added into 200 μL of breast milk, added with 2 mL of n-hexane and 2 mL of methyl alcohol, shaken the mixture for uniform mixing, shaken the same in a thermostatic water bath at 80° C. for 2 h, then cooled the obtained mixture to room temperature with running water, added with 2 mL of deionized water, shaken the mixture for uniform mixing, and subjected to refrigerated centrifugation at 5000 rpm for 5 min, and 1 mL of a supernatant (an ester layer solution) was put into a sample bottle for subsequent GC-MS. The sample was preserved at −20° ° C. before detection.

After the sample pretreatment was completed, detection was carried out by the GC-MS. In the present example, conditions of the chromatography were as follows: a chromatographic column was HP-88 GC column (100 m×0.25 mm, 0.20 μm); a temperature rising procedure included setting an initial temperature at 60° C., which was maintained for 5 min, increased to 160° C. at 8° C./min, then increased to 200° C. at 4° C./min and maintained for 5 min, and finally increased to 240° ° C. at 3° C./min and maintained for 5 min; a sample injector was Thermo Scientific TriPlus RSH automatic sample injector: a sample inlet temperature was 200° C.: a constant flow velocity mode was 1 mL/min: a sample injection volume was 1 μL: a split ratio was 10:1; and N₂ was used as a carrier gas.

Conditions of the mass spectrometry were as follows: an electron impact ion source was employed; an ion source temperature was 280° C.: a transmission line temperature was 240° C.: a quadropole temperature was 150° C.; and a quadropole mass scan range was m/z 35-400.

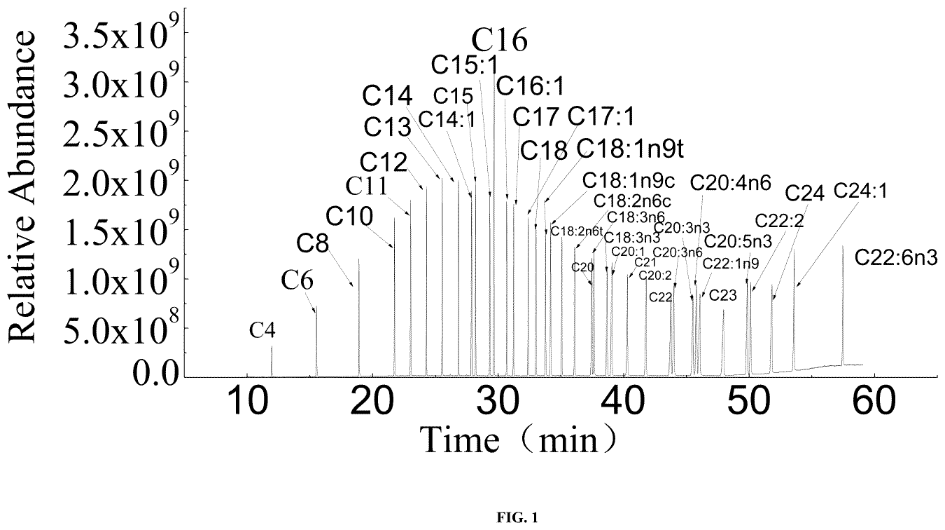

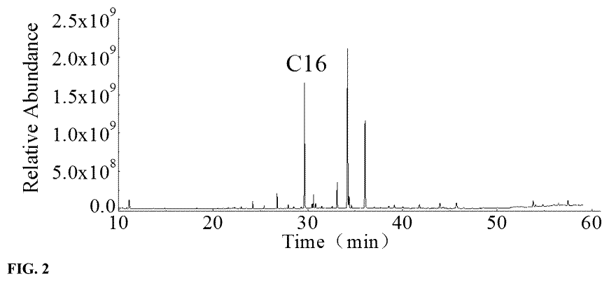

As an example, FIG. 1 is a total ion chromatogram of a methyl ester standard sample of 37 main fatty acids in measured breast milk; and FIG. 2 is a total ion chromatogram of No. 10256 breast milk sample during measurement.

After all the 254 samples were measured, 98 samples were randomly selected therefrom and subjected to correlation analysis on fatty acid data of the samples and Ya scores corresponding to the samples by SPSS21 statistical software. Fatty acids having a lowest significant difference at 95% confidence level (p<0.05) were selected as markers to form a marker set, and a standard curve and a data model were established through single factor analysis of variance and multiple linear regression analysis.

Analysis results of SPSS were shown in Table 1:

TABLE 1
Fatty Acid Analysis Results of SPSS

| Fatty Acid | B | Sig. |
|---|---|---|
| C18:3n6 | 0.115 | 0.01 |
| C18:2n6t | −1.001 | 0.024 |
| C20:3n3 | 0.054 | 0.024 |
| C18:3n3 | 0.004 | 0.014 |
| C18:2n6c | 0.003 | 0.015 |
| C20:4n6 | 0.065 | 0.023 |
| C20:3n6 | 0.03 | 0.049 |
| C22:6n3 | 0.009 | 0.049 |
| C20:5n3 | 0.003 | 0.049 |

According to Table 1, the established data model was as follows: Ya=0.115×C_{C18:3n6}−1.001×C_{C18:2n6t}+0.054×C_{C20:3n3}+0.004×C_{C18:3n3}+0.003×C_{C18:2n6c}+0.065×C_{C20:4n6}+0.03×C_{C20:3n6}+0.009×C_{C22:6n3}+0.003×C_{C20:5n3}, where the concentrations of each marker were in units of ppm.

Further, the Ya scores of the samples were counted and dimensions were set, with results shown in Table 2:

TABLE 2
Ya Scores and Dimensions

| Dimension | Ya |
|---|---|
| 0 | −10.5625 |
| 25% | 9.695722 |
| 50% | 17.29302 |
| 75% | 41.75746 |
| 100% | 104.4025 |

According to a Ya score obtained by model calculation, a level of the development of the communication ability of the infant belonging to the mother providing the sample among all the infants could be found correspondingly in the Table 2. When the level was too low, e.g., lower than 50% or 25%, it was recommended that timely dietary and nutritional interventions should be taken for the corresponding mother.

### Example 2

The remaining 156 samples of the 254 samples measured in Example 1 were taken as a validation set to validate the model obtained in Example 1. If the Ya score calculated with the model and the Ya score of the actual sample, the result was considered as correct.

Upon validation with the validation set, the accuracy rate of the model provided in Example 1 was 90.2%, and the sensitivity thereof was 95%. The above results showed that the model provided in Example 1 had good accuracy and sensitivity.

With the model, by measuring the contents of each marker fatty acid of the marker set in a breast milk sample or a dairy product sample for feeding, the development of the communication ability of an infant or a feeding object corresponding to the sample could be effectively predicted and evaluated so as to give suggestions and take interventions in terms of diet and nutrition timely.

The method and model provided in the present invention are low in cost, simple and effective, and suitable for large-scale evaluation, and have promising application prospects.

Finally, it should be noted that the foregoing examples are merely intended to explain the technical solutions of the present invention rather than to limit the same. Although the present invention is described in detail with reference to the foregoing examples, those of ordinary skill in the art should understand that they can still modify the technical solutions described in the foregoing examples, or make equivalent replacements to some technical features therein. These modifications or replacements do not cause the essence of the corresponding technical solutions to deviate from the spirit and scope of the technical solutions of the examples of the present invention.

1. A marker set for evaluating the development of an infant's communication ability, composed of the following fatty acids: C18:3n6, C18:2n6t, C20:3n3, C18:3n3, C18:2n6c, C20:4n6, C20:3n6, C22:6n3, and C20:5n3.

2. A method for evaluating the development of an infant's communication ability using the marker set according to claim 1, comprising the following sequential steps:
(1) collecting a sample, and pretreating the sample to obtain a sample to be measured;
(2) measuring the contents of each marker of the marker set in the sample to be measured using gas chromatography-mass spectrometry (GC-MS);
(3) calculating a score Ya of the sample from the measured contents of each marker; and
(4) evaluating the development of an infant's communication ability based on the score Ya.

3. The method according to claim 2, wherein in step (1), the sample is pretreated by performing methyl esterification on the sample using a hydrochloric acid-methanol method.

4. The method according to claim 3, wherein the hydrochloric acid-methanol method specifically comprises: adding 5 mL of a 0.5 mol/L hydrochloric acid-methyl alcohol solution into 200 μL of the sample, adding 2 mL of n-hexane and 2 mL of methyl alcohol thereinto, shaking the mixture for uniform mixing, shaking the same in a thermostatic water bath at 80° C. for 2 h, then cooling the obtained mixture to room temperature with running water, adding 2 mL of deionized water thereto, shaking the mixture for uniform mixing, followed by refrigerated centrifugation at 5000 rpm for 5 min, and putting 1 mL of a supernatant into a sample bottle, thereby obtaining the sample to be measured.

5. The method according to claim 2, wherein in step (2), parameters of GC of the GC-MS are as follows: HP-88 GC column having specifications of 100 m×0.25 mm and 0.20 μm is used; a temperature rising procedure comprises setting an initial temperature at 60° C., which is maintained for 5 min, increased to 160° ° C. at 8° C./min, then increased to 200° C. at 4° C./min and maintained for 5 min, and finally increased to 240° C. at 3° C./min and maintained for 5 min; a sample injector is Thermo Scientific TriPlus RSH automatic sample injector; a sample inlet temperature is 200° C.; a constant flow velocity mode is 1 mL/min; a sample injection volume is 1 μL; a split ratio is 10:1; and N₂ is used as a carrier gas.

6. The method according to claim 2, wherein in step (2), parameters of MS of the GC-MS are as follows: an electron impact ion source mode is employed; an ion source temperature is 280° C.; a transmission line temperature is 240° C.; a quadropole temperature is 150° C.; and a quadropole mass scan range is m/z 35-400.

7. The method according to claim 2, wherein in step(3), Ya is calculated by: Ya=0.115×C_{C18:3n6}−1.001×C_{C18:2n6t}+0.054×C_{C20:3n3}+0.004×C_{C18:3n3}+0.003×C_{C18:2n6c}+0.065×C_{C20:4n6}+0.03×C_{C20}:3n6+0.009×C_{C22:6n3}+0.003×C_{C20:5n3}, wherein the concentrations of each marker are in units of ppm.
